(19) United States
(12) Patent Application Publication
(10) Pub. No.: US 2016/0038660 A1
(43) Pub. Date: Feb. 11, 2016

(54) SUCTION AND IRRIGATION INSTRUMENT AND METHOD OF USE

(71) Applicant: NLT SPINE LTD., Kfar Saba (IL)

(72) Inventor: Oded LOEBL, Tel Mond (IL)

(21) Appl. No.: 14/821,991

(22) Filed: Aug. 10, 2015

Related U.S. Application Data
(60) Provisional application No. 62/035,463, filed on Aug. 10, 2014.

(51) Int. Cl. A61M 1/00

(52) U.S. Cl. CPC A61M 1/0064; A61M 1/0086; A61M 2039/082

(57) ABSTRACT

A surgical apparatus includes a first or suction tube, through which suction forces are applied through a tube-end opening along a longitudinal axis, and a second or irrigation tube, extending along Inc first tube, the second tube, through which irrigation fluid, typically liquids, travels, the second tube including apertures. through which irrigation fluid is expelled in an axial orientation different from the longitudinal axis. The suction and irrigation tubes form a circulation pathway for the introduced irrigation fluid and particulates, which are drawn into the suction tube by suction forces, tot properly clearing (flushing) the surgical site.

### CROSS REFERENCES TO RELATED APPLICATIONS

This application is related to and claims priority from commonly owned U.S. Provisional Patent Application Ser. No. 62/035,463, entitled: SUCTION AND IRRIGATION INSTRUMENT AND METHOD OF USE, filed on Aug. 10, 2014, the disclosure of which is incorporated by reference in its entirety herein.

### TECHNICAL FIELD

The present invention is directed to suction and irrigation devices, and more particularly to suction and irrigation devices for spinal surgery.

### BACKGROUND

The effective removal of debris in any lumbar interbody fusion procedure, such as Anterior Lumbar Interbody Fusion (AILF), Transforaminal Lumbar Interbody Fusion (TLIF), Lateral Lumbar interbody Fusion (LLIF), or other spine procedure (e.g., discectomy, corpectomy) is critical to the successful short and long term outcome of the procedure. Debris may be nucleus pulpous, bone chips and shavings, along with other or extraneous material (autogenous, biological and/or synthetic). There are mans instruments/devices that provide a tube connected to a suction pump for this purpose, There are also instruments (primarily in percutaneous and laproscopic surgeries) that have irrigation (water flow) as well as suction, and even have concurrent suction and irrigation. The water flow is usually axial to the tube and at a low pressure and/or low flow rate, for example, 1-2 bars and 2-3 cc per second, not greater than 5 bars and 5 cc per second,

### SUMMARY OF THE INVENTION

The invention is directed to devices and methods for debris removal during a surgical procedure by more aggressively ‘washing’ the desired region (surgical site) and concurrently removing (via integral, simultaneous suction, by a suction device) the loosened debris. The water or other fluid: gas or liquid) that is streamed, is directed at and around the area where the debris is located. The opening of the suction device is placed away from the water ports to create a circulation effect to improve the debris removal. For example, the direction of the fluid expulsion is along a different axis than the suction. Additionally, the fluid expulsion is performed by high pressure apertures or jet(s), which increase the fluid flow rate and pressure, for example, to high pressures, such as 5-10 bars and 5-10 cc per second flow rate, to dislodge debris.

The present invention is directed to a surgical apparatus comprising a first or suction tube, through which suction forces are applied, the first tube including a longitudinal axis and at least one opening along the longitudinal axis; and, a second or irrigation tube, extending along the first tube, the second tube, through which irrigation fluid, typically liquids, travels, the second tube including apertures oriented at least substantially perpendicular to the longitudinal axis. The first and second tubes are received in a body, which includes a channel through which the first tube couples to a suction source, and the second tube which includes a channel through which the second tube couples to an irrigation fluid source. With the suction and irrigation fluid sources operatively connected to the apparatus, the apparatus provides a circulation pathway for introduced irrigation fluid and particulates, which are brought into the suction tube by suction forces, for properly clearing (flushing) the surgical site.

Embodiments of the invention are directed to a surgical apparatus comprising: a first tube including a longitudinal axis and at least one opening along the longitudinal axis; and, a second tube extending along the first tube, the second tube including at least one aperture oriented at least substantially perpendicular to the longitudinal axis.

Optionally, the apparatus additionally comprises: a body for receiving the first tube and the second tube, the body configured for facilitating: 1) communication of the first tube with a suction source; and 2) communication of the second tube with an irrigation fluid source.

Optionally, the first tube coupled with the second tube provides a circulation pathway for irrigation fluid and particulates.

Optionally, the apertures are dimensioned to pressurize fluid upon the fluid exiting the apparatus.

Optionally, the second tube extends within the first tube at least substantially parallel to the first tube.

Optionally, the first tube and second tube are defined by a common wall extending substantially parallel to the longitudinal axis.

Optionally, the first tube extends at least within the second tube.

Optionally, the first tube and second tube are concentric.

Optionally, the second tube includes two lumens oppositely disposed with respect to the first tube.

Optionally, the first tube extends beyond the second tube, and the first tube includes a lip defining a protrusion from the first tube, the lip proximate to the at least one opening along the longitudinal axis.

Optionally, the at least one opening along the longitudinal axis includes one opening.

Optionally, the apparatus additionally comprises: an opening in the first tube oriented substantially perpendicular to the longitudinal axis.

Optionally, the first and second tubes are removably attachable from the body.

Embodiments of the present invention are also directed to a method for performing a surgical procedure. The method comprises: obtaining an apparatus comprising: a first tube including a longitudinal axis and at least one opening along the longitudinal axis, the first tube coupled to a suction source; and, a second tube extending along the first tube, the second tube including at least one aperture oriented at least substantially perpendicular to the longitudinal axis, the second tube coupled to an irrigation fluid source; positioning the apparatus with respect to the surgical site; irrigating the surgical site by providing irrigation fluid through the at least one aperture of the second tube at an axial orientation different from the axial orientation of the suction through the first tube from the suction source; and, providing suction to the surgical site by applying suction from the suction source through the first tube, to clear irrigation fluid and debris from the surgical site.

Optionally, the first tube and the second tube of the apparatus couple to define a circulation pathway for irrigation fluid and surgical debris.

Optionally, the irrigation fluid is expelled through the at least one aperture at a greater pressure than which the irrigation fluid was delivered.

Optionally, the irrigation and suction operate contemporaneously.

Optionally, the irrigation and suction operate simultaneously.

Optionally, the irrigation and suction operate at different times.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

Attention is now directed to the drawings where like numerals or characters represent like or corresponding components. In the drawings:

FIG. 1A is a top view of an apparatus of the present invention positioned inside a conduit;

FIG. 1B is a longitudinal cross sectional view of the apparatus of FIG. 1A;

FIG. 2 is a perspective view of an apparatus of the present invention;

FIG. 3 is an axial view of the apparatus of FIG. 2;

FIG. 4 is a perspective view of the distal end of the apparatus of FIG. 2;

FIG. 5 is a longitudinal cross sectional view of the apparatus of FIG. 2 showing the suction and irrigation pathways;

FIG. 6A is a perspective view of an alternative apparatus;

FIG. 6B is a perspective view of the distal end of the apparatus of FIG. 6A;

FIG. 6C is a cross sectional view of the tube member taken along line 6C-6C of FIG. 6B;

FIG. 7 is a cross sectional perspective view of another alternative tube member;

FIGS. 8A, 8B and 8C are perspective views of the distal ends of the tube member of FIG. 6A showing various aperture arrangements and aperture configurations;

FIGS. 9A, 9B and 9C are perspective views of the distal ends of the tube member of FIG. OA showing various aperture bore configurations;

FIG. 10A is an alternative apparatus of the present invention;

FIG. 10B is a longitudinal cross-sectional view of the tube member of FIG. 10A;

FIG. 11A is a side view of an alternative apparatus in accordance with the present invention;

FIG. 11B is a longitudinal cross sectional view of the apparatus of FIG. 11A;

FIG. 11C is a cross sectional view taken along line 11C-11C of FIG. 11B;

FIG. 11D is a cross sectional view taken along line 11D-11D of FIG. 11B;

FIGS. 12A-12C are perspective views of another alternative apparatus of the present invention;

FIGS. 13A-13C are perspective view showing an exemplary operation of the apparatus of the present invention; and,

FIGS. 14A and 14B are perspective view showing an alternative apparatus performing processes in accordance with the present invention.

### DETAILED DESCRIPTION OF THE DRAWINGS

The present invention provides an apparatus which creates a circulation, pathway for the flow of irrigation fluid to a surgical site, and suction for the fluid and debris from the surgical site, away from the surgical site. The fluid flow and suction of the circulation pathway of the apparatus are performed contemporaneously, and typically simultaneously. Additionally, the apparatus is configured to control the fluid flow pattern out of the apparatus to the surgical site, based on the numbers of, and shapes and dimensions of the bores and openings for the apertures to the apparatus.

Throughout this document, reference is made to directions and orientations liar the apparatus of the invention as well as any devices, components and the like used in association therewith. These directions and orientations are exemplary, in order to describe the invention, and include directions such as “proximal,” “proximally” and all forms and derivatives thereof, and “distal”, and “distally” and all forms and derivatives thereof, as well as “posterior” and “anterior” and all forms and derivatives thereof.

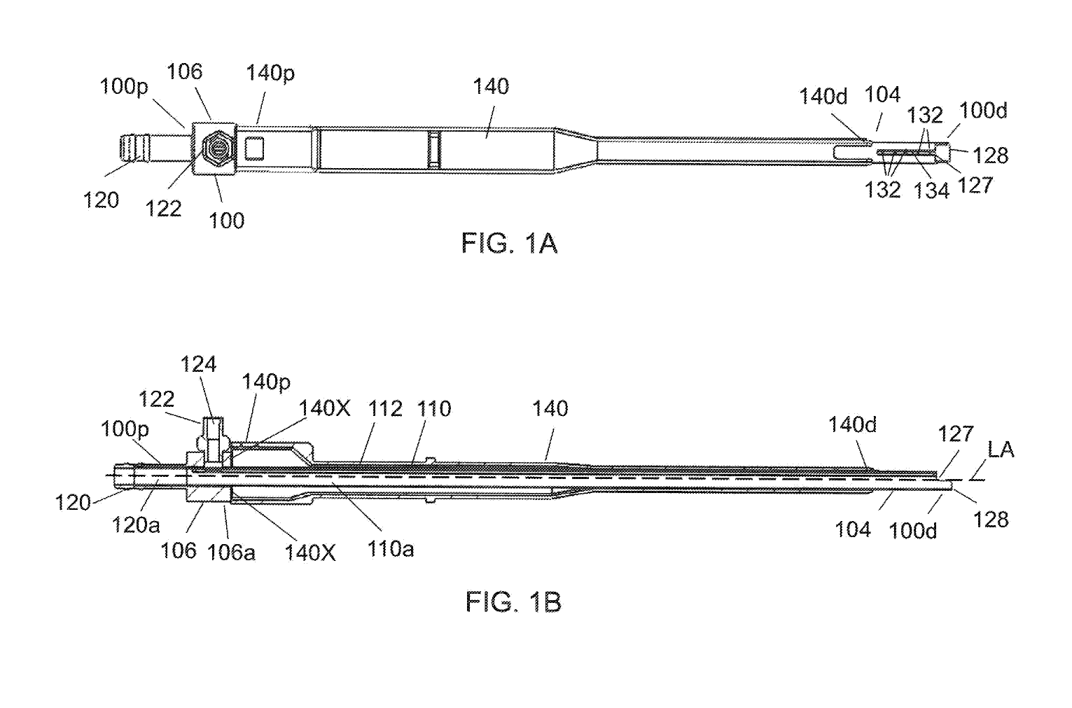

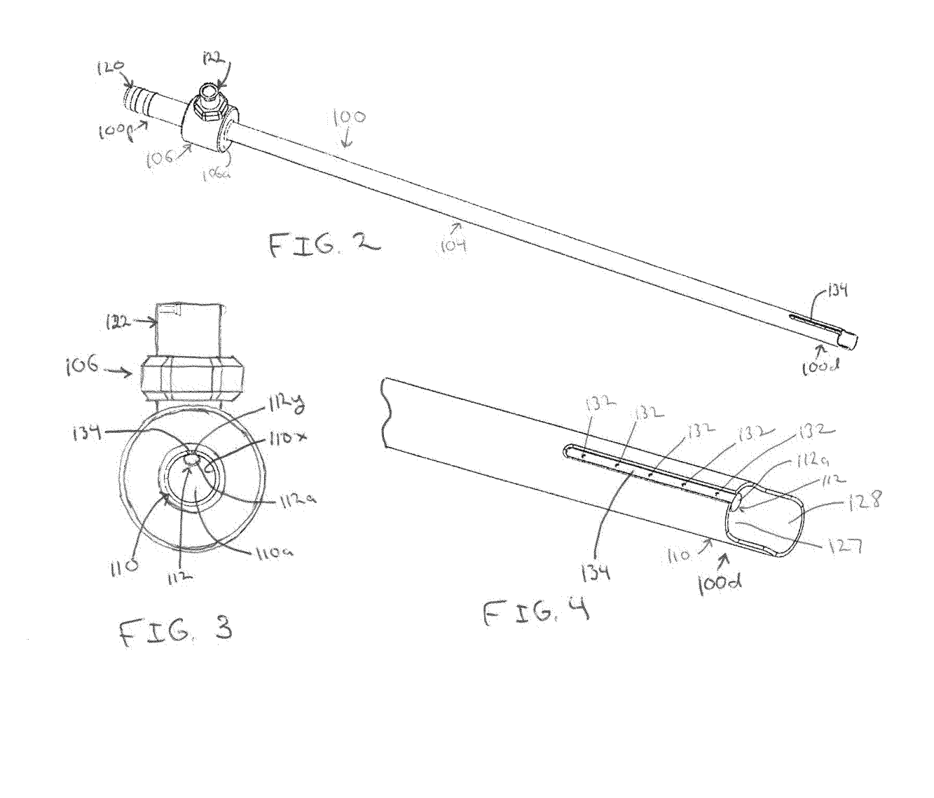

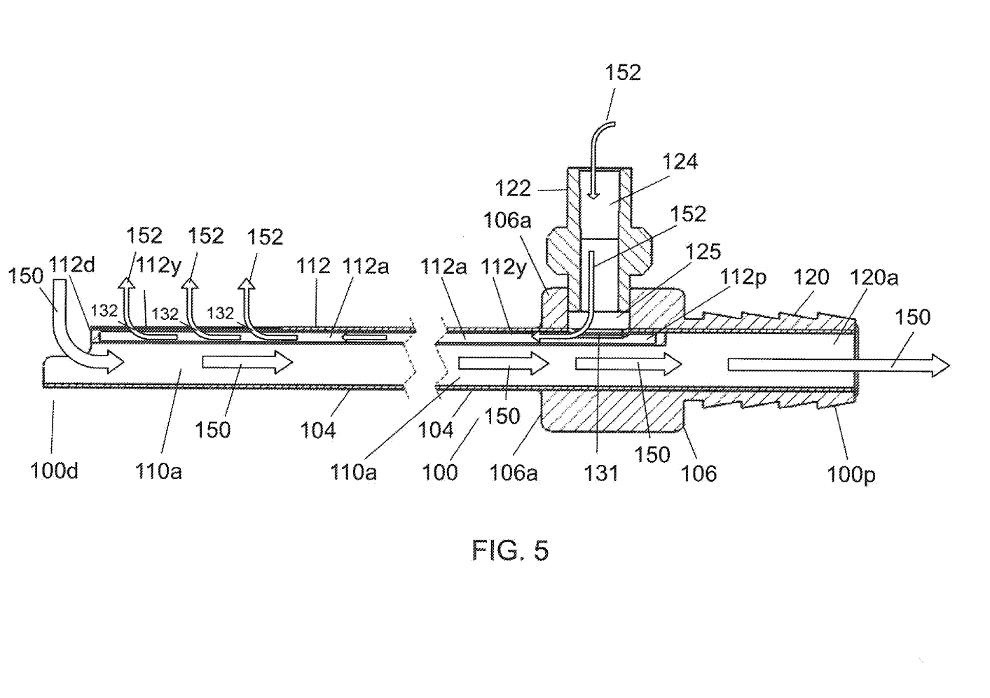

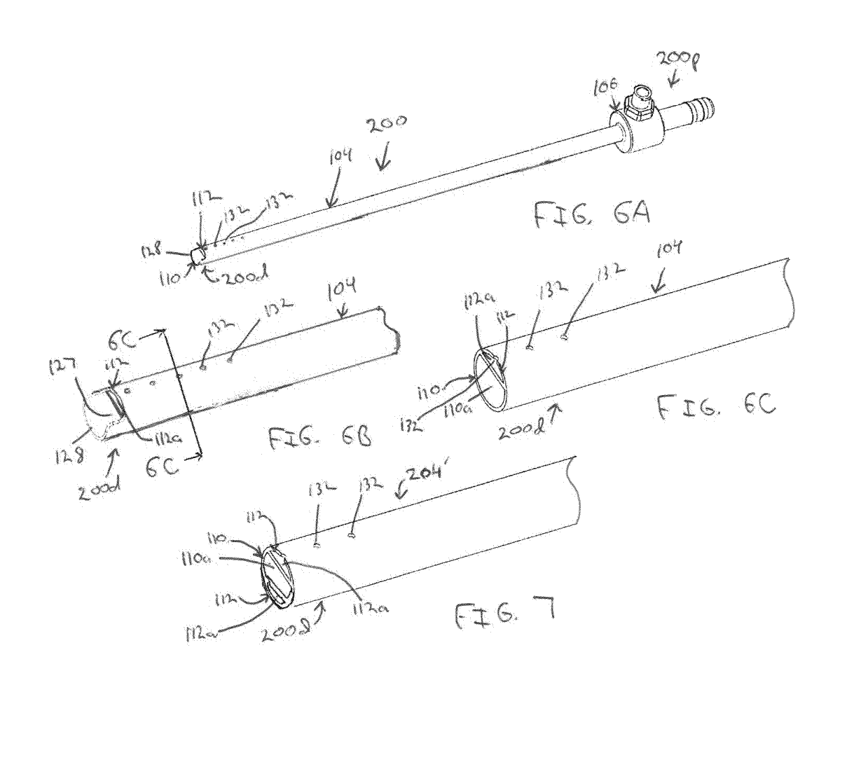

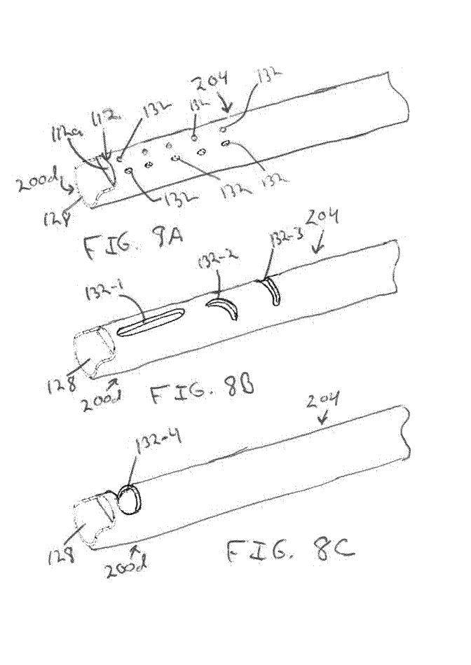

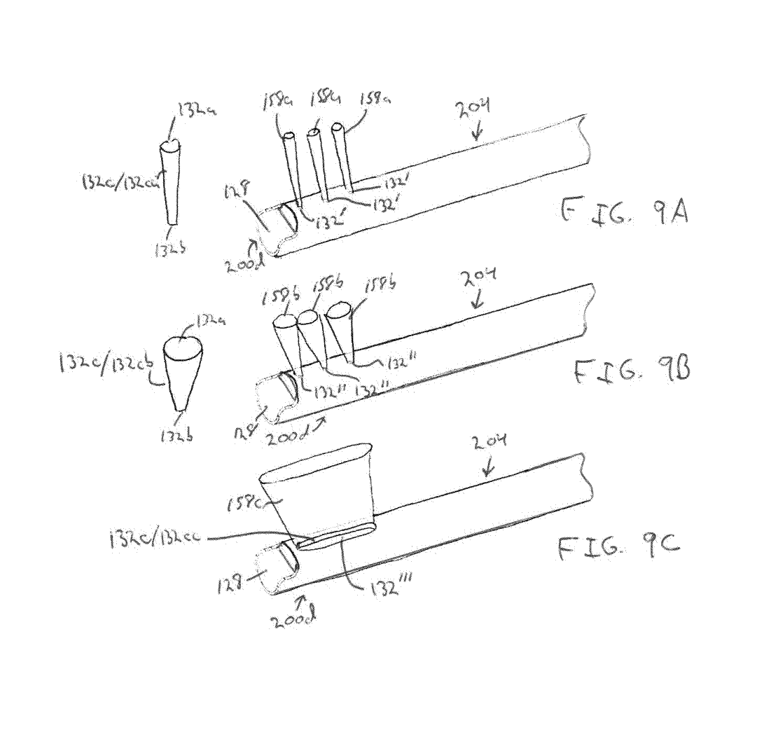

FIGS. 1A and 1B show an apparatus 100 as a tube member (“apparatus” and “tube member” used interchangeably herein) of the present invention. The tube member 100 includes a tubular or tube portion 104, extending from a collar or body 106. The tube member 100 is a stand-alone device, but is usable with other deuces such as conduits 140, retractors and other surgical tubes, cannulas, trocars, introducers, and the like, depending on the surgical application. The tube member 100 extends through the conduit 140, with the proximal 100ₚ and distal 100d ends of the tube member 100 extending beyond the respective proximal 140p and distal 140d ends of the conduit 140.

Turning also to FIGS. 2, 3 and 4, the tube member 100 is formed of the tubular portion 104, of single or multiple tubes, with single or multiple lumens, and a longitudinal axis LA extending through the center of the tubular portion 104 (FIG. 1B). The tubular portion 104 extends distally from a collar 106. The collar 106 is at the proximal end of the tube member 100. The tubular portion 104, for example, removably attaches to the collar 106, for example, by friction fits such as correspondingly threaded portions and the like. This removable attachment allows for separation and cleaning/sterilization of the tubular portion 104 and the collar 106. However, the attachment may also be fixed or permanent. The collar 106 includes a surface 106a, which as shown here, abuts a solid portion 140x of the conduit 140, preventing the collar 106 from moving into the conduit 140.

For example, the tubular portion 104 include a suction 110, or main tube, with an inner lumen 110a, and an irrigation 112, or secondary tube, with an inner lumen 112a, The collar 106 includes a connector 120, with its lumen 120a aligned with the lumen 110a of the suction tube 110, to form a suction pathway, and another connector 122 (with a lumen 124), which aligns with a channel 125 in the collar 106, which in turn, aligns with the lumen 112a of the irrigation tube 112, to create an irrigation fluid, e.g., liquid or gas, pathway, for the apparatus 100. The suction pathway and the irrigation pathway operate in a combined and coordinated manner to define a circulation pathway (path or circuit) for fluid, including irrigation fluid, body fluids, and debris, from surgical sites. For example, when the suction tube 110 is coupled with a suction source, and the irrigation tube 112 is coupled with an irrigation fluid source, suction and irrigation operate contemporaneously, and for example, simultaneously, defining the aforementioned circulation pathway. However, the suction and irrigation can operate separately, at different times, such as at intervals of suction and irrigation, to also define the aforementioned circulation pathway. The suction and irrigation can also be operated randomly, for example, each being manually activated by the surgeon. The suction and irrigation sources may be connected to computerized devices to control their operation, and accordingly, control the flow of the circulation path.

The suction tube 110 extends from the connector 120 of the collar 106, at the proximal end 100p of the tube member 100, to an opening 127 and a lip 128, at the distal end 100d of the tube member 100. The opening 127 is an intake opening for suction and, for example, is axially oriented, along the longitudinal axis LA of the tube member 100 and tubular portion 104. The protruding lip 128 allows for capture of debris proximate to the opening 127, prior to the debris being pulled into the opening 127 of the suction tube 110 by the forces of the suction source.

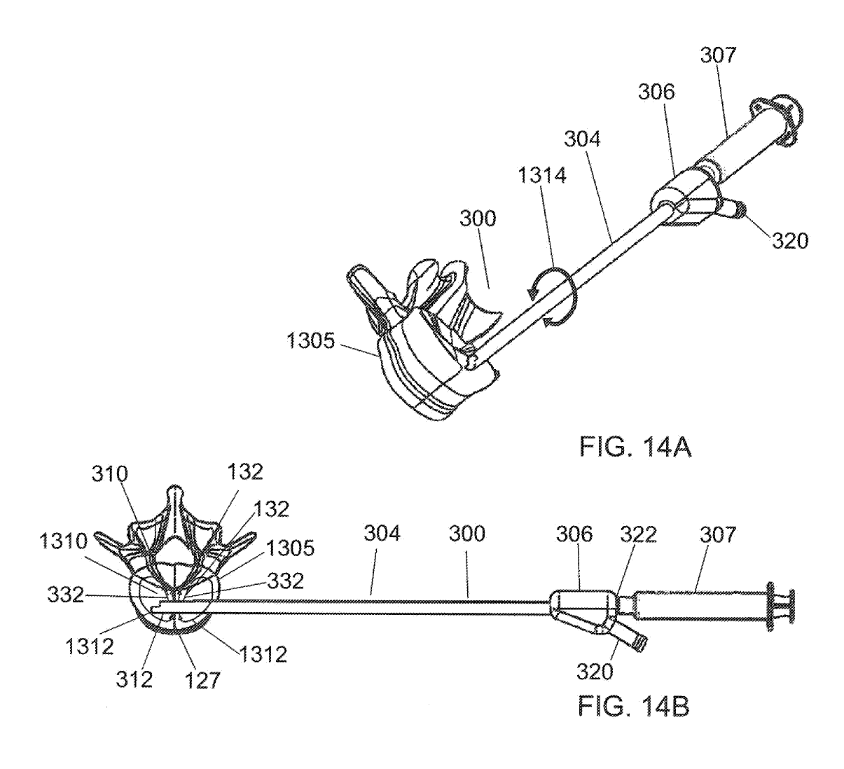

Alternately, for this type of tubular portion 104, the suction tube 110 may include one or more openings 127′ (suction intake openings-only one shown) laterally positioned perpendicularly or substantially perpendicular to the longitudinal axis LA, in the side of the suction tube 110 (at the distal end 100d of the tube member 100), as shown in FIG. 14B. This alternative side (lateral) opening(s) 127′ may alone serve as the suction opening or may be in addition to the distal (axial) opening 127. As shown in FIG. 14B, for example, the opening 127′ shown opposite (approximately 180 degrees) from the apertures or ports 132 of the irrigation tube 112 (these apertures 132 are also laterally oriented with respect to the longitudinal axis LA), but can be anywhere along the distal end of the suction tube 112.

At the connector 120, suction sources, such as suction tubes to suction pumps (e.g., manual, hydraulic, pneumatic, electric) are attached. The connector 120 is, for example, in the form of a nipple. The suction tube 110 is open at the distal end 100d of the tube member 100, to allow fluid and particulates, e.g., debris, to be pulled proximally (toward the proximal end 100p of the apparatus 100) by the suction source (not shown) through the lumen 110a of the suction tube 110 and the lumen 120a of the connector 120, to define a suction path, which in turn, forms a portion of the circulation path, as represented by the arrows 150 of FIG. 5.

The irrigation tube 112 extends, for example, within the suction tube 110, e.g., the lumen 110a thereof. The irrigation tube 112 is, for example, attached along the inner wall 110x of the suction tube 110. The irrigation tube 112 is for example, dosed at its proximal 112p and distal 112d ends (FIG. 5), and includes apertures 131 (FIG. 5), 132, which extend through the wall 112y of the tube 112. At the proximal end 112p of the irrigation tube 112, is, for example, a single aperture 131, which aligns with the channel 125 and the lumen 126 of the connector 122. The distal end 112d of the irrigation tube 112 includes aperture's 132, which include openings 132a, 132b with a bore or jet 132c extending between the openings 132′, 132″ and 132′, as shown in detail in FIGS. 9A-9C. The apertures 132 are open to the ambient environment, at a cut-out portion 134 of the suction tube 110. The apertures 132 are, for example, laterally positioned in the tube member 100, so as to be oriented perpendicularly or substantially perpendicular to the longitudinal axis LA (FIG. 1B) of the tube member 100, such that fluid flows through the apertures 132 and out of the irrigation tube in a direction perpendicular or substantially perpendicular to the suction direction, in the suction tube 110. The apertures 132 are formed in a peripheral wall of the irrigation tube 112, so as to face outwards from the longitudinal axis LA. The apertures 132, are, for example, such that the opening 132a, 132b shapes and diameters, as well as bore 132e shapes and dimensions can be adjusted to accommodate the desired fluid pressure and flow rates of fluid leaving the irrigation tube 112, for example, to the surgical site.

Alternately, should the cut-out portion 134 not be present, the apertures 132 can extend through the suction tube 110, so that fluid from the irrigation tube 112 can be delivered to and reach the ambient environment (e.g., the surgical site). Accordingly, an irrigation pathway, which is part of the circulation path for the irrigation fluid, is formed from the connector 122 (via its lumen 124), through the channel 125, through the lumen 112a of the tube 112, to the apertures 132, at the distal end 100d of the tube member 100, as shown by the arrows 152 in FIG. 5.

The connector 122, is, for example, a leuer connector, so as to connect to a singe or other pressurizeable fluid source, including pressurized fluid sources, manual or automatic pumps.

The suction 110 and irrigation 112 tubes of the tubular portion 104 are shown as cylinders of circular cross-section, and made of materials such as surgical grade metals, polymers and the like, so as to be rigid and/or flexible. The collar 106 and is also made of the aforementioned materials. The tubular portion 104 and collar 106 may also be permanently attached and in this case joined by conventional materials joining techniques. However, the tubes 110, 112 can be square, rectangular, rounded, or triangular in cross section, or any combination thereof, with the collar 106 configured accordingly, to receive the tubular portion 104 (which includes the tubes 110, 112).

FIGS. 6A-6C show an alternative tube member 200. In this alternative tube member 200, the suction tube 110 and the irrigation tube 112 include D-shaped lumens 110a, 112a, shown in detail in FIG. 6C. In the tubular portions 204, the central lumen 110a terminates in an axial opening 127, while perpendicularly oriented apertures 132 extend from lumen 112a, through the tube member 200 to the ambient environment.

FIG. 7 shows another alternative tubular portion 204′ of the tube member 200 at a distal end 200d, which includes three lumens, a central lumen 110a of the suction tube 110, and lateral lumens 112a defining the irrigation tube 112 or tubes 112. White apertures 132 are shown extending from one of the lateral lumens 112a, apertures, identical or similar to the lumens 132, may also be positioned so as to extend from to the other lateral lumen 112a, either alone or in addition to the lumens 132.

FIGS. 8A, 8B and 8C show various aperture 132 arrangements and opening 132a, 132b shapes for the tubular portions 204, which are applicable to all tubular portions 104, 304, 404, 504 disclosed herein. in FIG. 8A, the apertures 132 are arranged radially and/or axially, and are in communication with the lumen 112a of the irrigation tube 112. While the shapes for the openings 132a, 132b of the apertures 132 are typically identical or similar, they may be different due to, for example, the shaping and dimensioning of the corresponding bore 132c of the aperture 132 (FIGS. 9A-9C).

In FIG. 8B, the apertures 132-1, 132-2, 132-3, may be one or more, and of shapes such as, for example, linear 132-1, rounded 132-2, radial 132-3, and combinations thereof. These apertures 132p, 132q, 132r are in communication with the lumen 112a of the irrigation tube 112. FIG. 8C shows a single large aperture 132-4, which is in communication with the lumen 112a of the irrigation tube 112.

FIGS. 9A, 9B and 9C show various aperture bore 132c cross sectional shapes for the tubular portion 204, as an example for all tubular portions 104, 304, 404, 504, for the example apertures 132′, 132″, 132″′. The bore 132c cross sections are narrow 132ca, as shown. in FIG. 9A, wide 132cb, as shown in FIG. 9B and long (lengthwise) 132ce, as shown in FIG. 9C. The resultant spray or jet streams 158a, 158b, 158c are shown for each aperture 132 (jet) are also displayed. The aforementioned apertures or jet(s) 132 function to increase the fluid flow rate and pressure, for example, to high pressures, such as 5-10 bars and 5-10 cc per second flow rate, to dislodge debris.

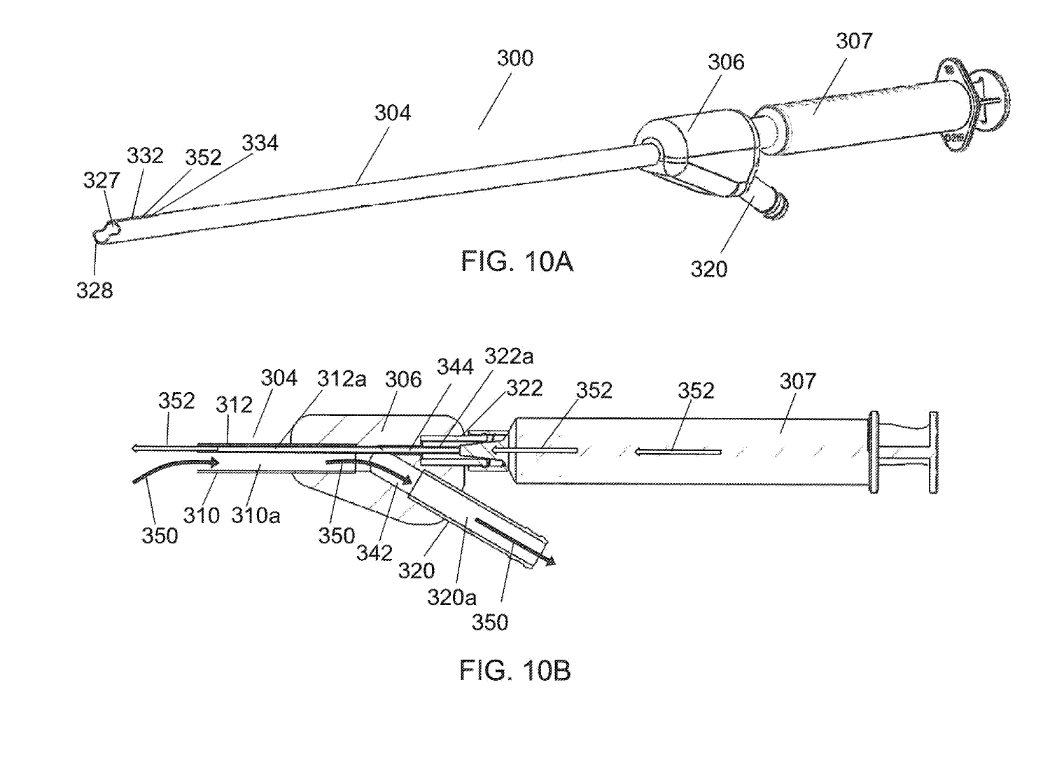

FIGS. 10A and 10B show an alternative apparatus 300 formed of a tubular or tube portion 304, extending from a body 306. For example, the tubular portion 304 is removably attachable to the body 306, to allow for separation and elevating/sterilization, but can also be a permanent or fixed attachment. The tubular portion 304 is similar to tubular portion 104 with identical or similar components to those of the tubular portion 104 numbered in the corresponding “300s.” For example, with the suction tube 310 and irrigation tube 312 are in accordance with the descriptions for the corresponding suction 110 and irrigation 112 tubes provided above, except where indicated, and are constructed and arranged in accordance with the corresponding suction 110 and irrigation 112 tubes, as detailed above.

The body 306 includes connectors 320, 322, which extend from the body 302, for example, proximally. The connectors include one or more suction connectors, represented by suction connector 320, to which a suction source (as detailed above) connects, and irrigation connectors, represented by irrigation connector 322, to which an irrigation source, such as a syringe 307, or other irrigation thud source, is connected.

The tubular portion 304, is received, by and aligns in the body 306, such that the suction tube 310, e.g., lumen 310a, aligns with a suction channel 342, which is in turn, aligned with the suction connector 320 (the lumen 320a) thereof, and a suction source (not shown). This alignment creates a suction pathway (shown by arrows 350) for suction air, particulates and liquids, from the ambient environment, typically a surgical site. The irrigation tube 312, e.g., lumen 312a, aligns with an irrigation channel 344, which in turn, aligns with the irrigation connector 322 (lumen 322a), and the syringe 307, the irrigation fluid source, to provide an irrigation pathway, shown by arrows 352, for irrigating liquids, gasses and the like. The suction pathway and irrigation pathway combine to for the aforementioned circulation path, for irrigating a surgical site and clearing the site of fluid and debris.

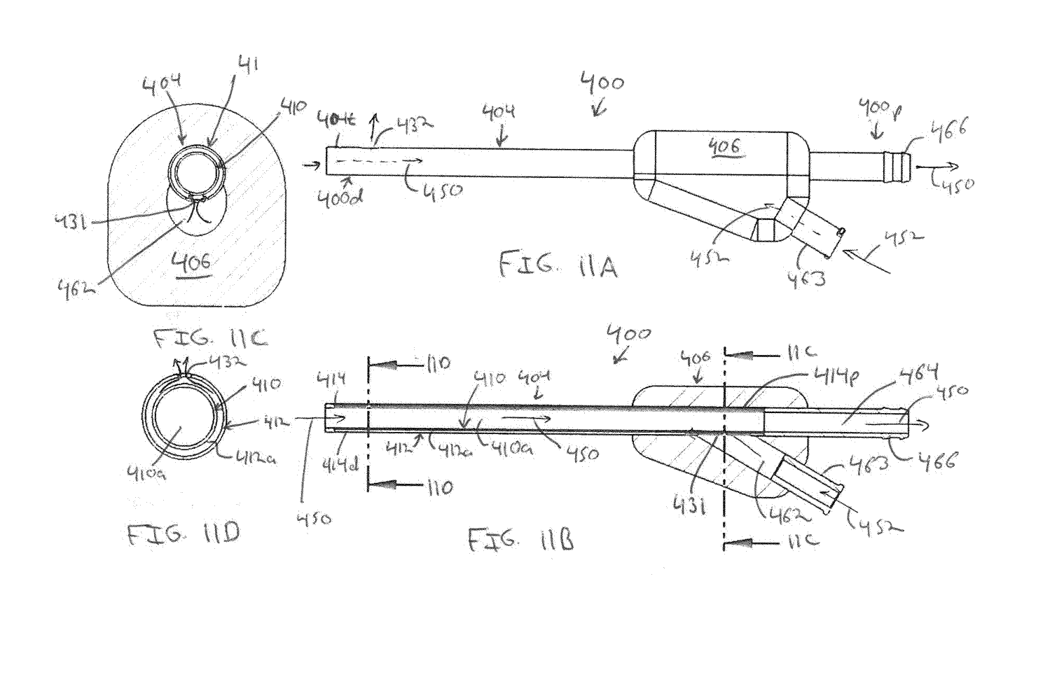

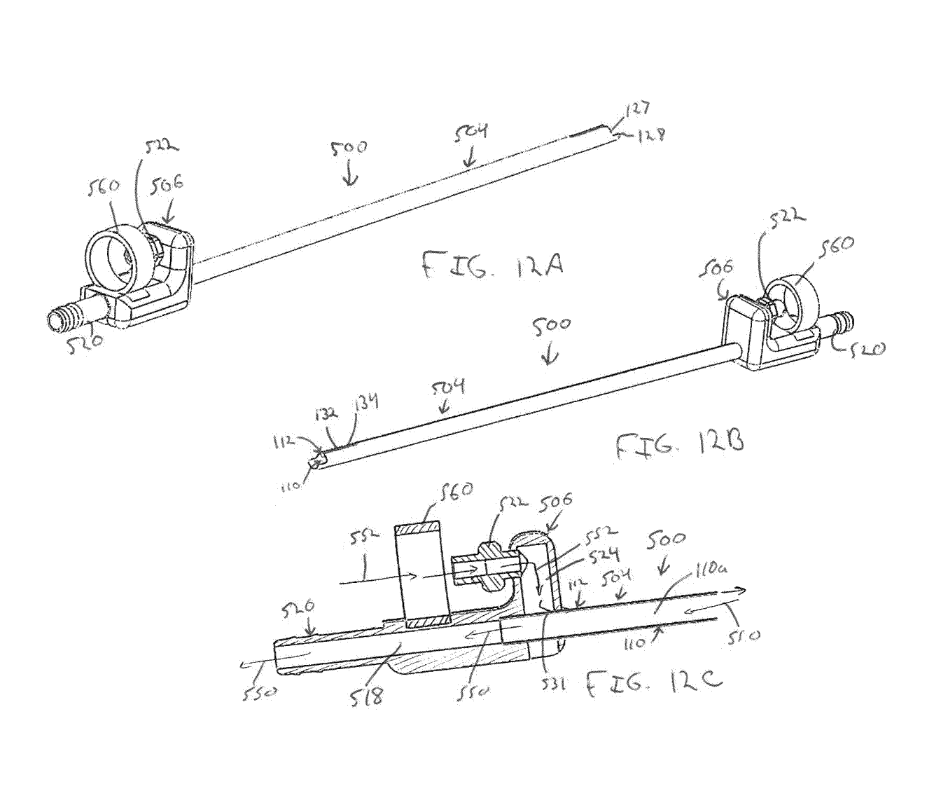

FIGS. 11A-11D show an alternative embodiment apparatus 400, for example a tube member. in the tubular portion 404 of the apparatus 400, the irrigation 412 and suction 410 tubes are concentric at the distal end 400d of the apparatus, with the suction tube 410 being the inner tube and the irrigation tube 412, being the outer tube. The lumen 412a between the irrigation tube 412, the outer tube, and the suction tube 410, the inner tube, is for introducing irrigation fluid to the surgical site. Suction is through the lumen 410a of the suction or inner tube 410. A body 406, similar to the body 306, receives the tubular portion 404, for example in a removably attachable engagement of a fixed or permanent engagement as detailed above for the body 306.

The suction (inner) tube 410 attaches to irrigation (outer) rube 412 at bands 414p, 414d both the proximal and distal ends of the tubular portion 404. These bands 414p, 414d seal the tubular portion 404 in a fluid tight manner. Between the bands 414p, 414d, the lumen 412a is open to allow for the passage of irrigating fluids, e.g., liquids and gasses. The irrigation (outer) tube 412 at the proximal end 400p of the tube member 400, includes an opening 431 for receiving irrigation fluid from a fluid source, as detailed above, through a channel 462 of the body 406, which extends into a connector 463 for the irrigation fluid source. The irrigation (outer) tube 412 includes one or apertures 432, in accordance with those detailed above, at the distal end 400d of the tube member 400, such that irrigation fluid flows out of the irrigation tube 412 (e.g., to the ambient environment/surgical site laterally. The irrigation fluid travels as per arrows 452 defining the irrigation fluid pathway, which is a portion of the circulation pathway.

The suction (inner) tube 410 is open at the tip 404t, at the distal end 400d of the apparatus (the tube member) 400. The tip 404t while shown as circular, or rounded, may be other suitable shapes such as flat, contoured, oval, formed, and the like. The lumen 410a of the suction tube 410 aligns with a channel 464, partly in the body 406 and partly in a connection nipple 466, which connects to a suction source (not shown), as detailed above. The suction is in the direction of arrows 450, defining the suction pathway, which is a portion of the circulation pathway.

FIGS. 12A-12C show another embodiment apparatus 500, for example a tube member, including a tubular portion 504 received in a body 506, in a removably attachable or permanent engagement as detailed above. The tubular portion 504 is identical or similar to tubular portions 104 and 304, as detailed above. The tubular portion 504 includes the suction tube 110 and irrigation tube 112, as shown in FIGS. 1A, 1B and 2-5 and described above.

The body 506 includes a channel 518, which aligns with the lumen 110a of the suction tube 110, and a nipple 520, which includes a portion of the channel 518. A suction source, as detailed above, connects to the nipple 520, to provide the suction pathway (arrows 550), of the circulation pathway, for the tube member 500.

Through opening 131, the irrigation tube 112 of the tubular portion 504 receives irrigation fluid, for example, from a syringe, similar to the syringe 307 as detailed above. The body 506 includes a connector 522 to which the syringe (not shown) connects and a channel 524 through which fluid flows through the body 506, from the syringe (not shown) to the opening 531, so as to enter the irrigation tube 112 for movement therethrough, to and through the apertures 132 to the surgical site. This irrigation flow pathway, of the circulation pathway, is indicated by the arrows 552. A ring 560, joined to the body 506, serves as a syringe holder.

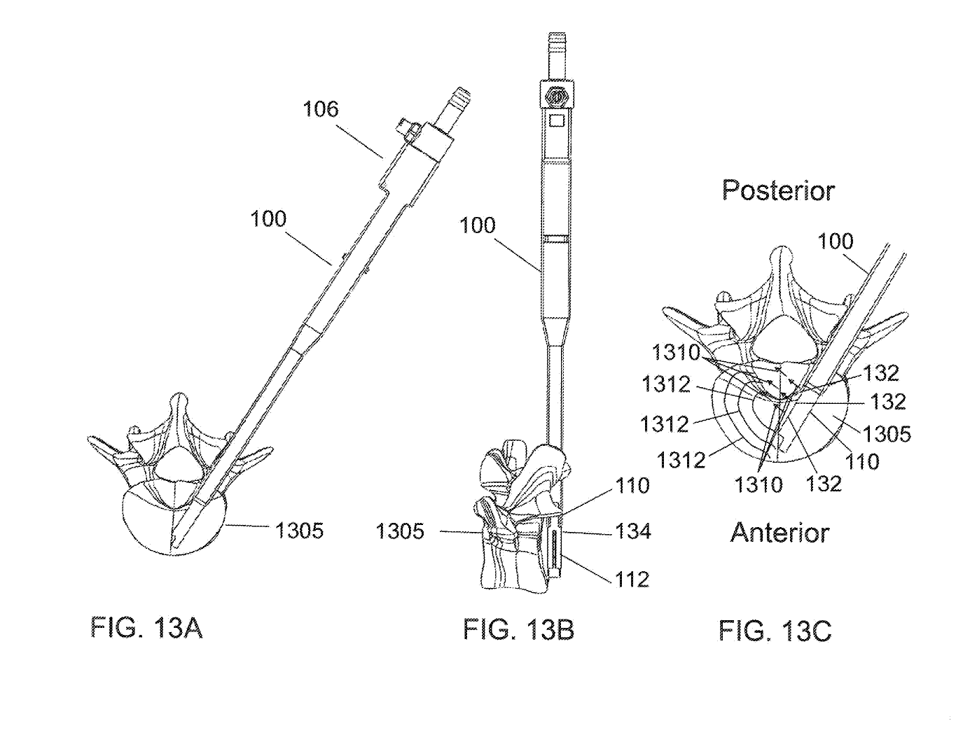

FIGS. 13A-13C show an operational mode for the apparatus 100, exemplary of all apparatus 200, 300, 400, 500, of the present invention. In FIGS. 13A and 13B, the apparatus 100 has been disposed along a vertebrae 1305 by surgical accessing techniques. The apparatus 100 may be positioned or rotated to a position of the surgical site, where irrigation and debris removal are desired. The irrigation tube 112, at its apertures 132, is proximate to the surgical site, so that the irrigation fluid is sprayed at high pressures through the apertures 132 at the posterior contralateral side of the vertebrae (arrows 1310 of FIG. 13C). The suction forces (arrows 1312 of FIG. 13C) pull the irrigation fluid debris and other loose substances and liquids into the suction tube, with the debris and the like collecting at the lip 128. With the irrigation and suction occurring simultaneously, the circulation path is created for irrigation and suction.

As shown in FIG. 14, in apparatus 300 (FIGS. 10A and 10B and detailed above), performing the procedure of FIGS. 13A-13C, the tubular portion 304 can be rotated (arrow 1314) to direct the irrigation fluid stream to the desired areas of the surgical site.

FIG. 14B shows the process of FIGS. 13A-13C and 14A, except that suction is through a suction opening 127′ at the side of the suction tube 310. The opening 127′ for suction is, for example, approximately 180 degrees from the apertures 332 through which fluid exits the irrigation tube 312.

While the apparatus and methods detailed herein have been described for procedures, such as those to be performed on vertebrae and associated tissues, the apparatus and methods are also employable in other types of surgeries, both human and animal. It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A surgical apparatus comprising:
a first tube including a longitudinal axis and at least one opening along the longitudinal axis: and,
a second tube extending along the first tube, the second tube including at least one aperture oriented at least substantially perpendicular to the longitudinal axis.

2. The apparatus of claim 1, additionally comprising: a body for receiving the first tube and the second tube, the body configured for facilitating: 1) communication of the first tube with a suction source; and, 2) communication of the second tube with an irrigation fluid source.

3. The apparatus of claim 2, wherein, the first tube coupled with the second tube provides a circulation pathway for irrigation fluid and particulates.

4. The apparatus of claim 2, wherein the apertures are dimensioned to pressurize fluid upon the fluid exiting the apparatus.

5. The apparatus of claim 2, wherein the second tube extends within the first tube at least substantially parallel to the first tube.

6. The apparatus of claim 5, wherein the first tube and second tube are defined b a common wall extending substantially parallel to the longitudinal axis.

7. The apparatus of claim 2, wherein the first tube extends at least within the second tube.

8. The apparatus of claim 7, wherein the first tube and second tube are concentric.

9. The apparatus of claim 7, wherein the second tube includes two lumens oppositely disposed with respect to the first tube.

10. The apparatus of claim 7, wherein the first tube extends beyond the second tube, and the first tube includes a lip defining a protrusion from, the first tube, the lip proximate to the at least one opening along the longitudinal axis.

11. The apparatus of claim 10, wherein the at least one opening along the longitudinal axis includes one opening.

12. The apparatus of claim 10, additionally comprising: an opening in the first tube oriented substantially perpendicular to the longitudinal axis.

13. The apparatus of claim 2, wherein the first and second tubes are removably attachable from the body.

14. A method for performing a surgical procedure comprising:
obtaining an apparatus comprising:
a first tube including a longitudinal axis and at least one opening along the longitudinal axis, the first tube coupled to a suction source; and
a second tube extending along the first tube, the second tube including at least one aperture oriented at least substantially perpendicular to the longitudinal axis, the second tube coupled to an irrigation fluid source;
positioning the apparatus with respect to the surgical site;
irrigating the surgical site by providing irrigation fluid through the at least one aperture of the second tube at an axial orientation different from the axial orientation of the suction through the first tube from the suction source; and,
providing suction to the surgical site by applying suction from the suction source through the first tube, to clear irrigation fluid and debris from the surgical site.

15. The method of claim 14, wherein the first tube and the second tube of the apparatus couple to define a circulation pathway for irrigation fluid and surgical debris.

16. The method of claim 14 wherein the irrigation fluid is expelled through the at least one aperture at a greater pressure than which the irrigation fluid was delivered.

17. The method of claim 14, wherein the irrigation and suction operate contemporaneously.

18. The method of claim 14, wherein the irrigation and suction operate simultaneously.

19. The method of claim 14, wherein the irrigation and suction operate at different times.
